(19) United States
(12) Patent Application Publication
(10) Pub. No.: US 2020/0253468 A1
(43) Pub. Date: Aug. 13, 2020

(54) OPHTHALMIC INSTRUMENT, IMAGE GENERATION DEVICE, PROGRAM, AND OPHTHALMIC SYSTEM

(71) Applicant: NIKON CORPORATION, Minato-ku, Tokyo (JP)

(72) Inventors: Ken TOMIOKA, Yokohama-shi (JP); Shota MIYAZAKI, Fujisawa-shi (JP); Hideki OBARA, Kawasaki-shi (JP)

(73) Assignee: NIKON CORPORATION, Minato-ku, Tokyo (JP)

(21) Appl. No.: 16/642,796

(22) PCT Filed: Aug. 24, 2018
(86) PCT No.: PCT/JP2018/031384

§ 371 Date: Feb. 27, 2020

(30) Foreign Application Priority Data
Aug. 31, 2017 (JP) 2017-167927

(51) Int. Cl. A61B 3/00; A61F 2/16; A61B 3/18

(52) U.S. Cl. CPC A61B 3/0008; A61F 2/16; A61B 3/18; A61B 3/005; A61B 3/0058

(57) ABSTRACT

Provided are an ophthalmic instrument, an image generation device, a program, and an ophthalmic system capable of allowing a patient to directly experience and confirm how vision will actually appear following surgery.

The ophthalmic instrument includes a light source, an optical system that guides light emitted from the light source onto a retina of a subject eye, a communication section that receives a simulation image generated based on optometry information for the subject eye and intraocular lens information relating to an intraocular lens prescribable for the subject eye, the simulation image corresponding to how vision would appear in a case in which the intraocular lens is prescribed for the subject eye, and a control section that controls the light source and the optical system such that the simulation image received by the communication section is projected onto the retina.

### TECHNICAL FIELD

The technology disclosed herein relates to an ophthalmic instrument, an image generation device, a program, and an ophthalmic system.

### BACKGROUND ART

In the present specification, ophthalmology indicates the field of medicine that handles eyes. In the present specification, for ease of explanation intraocular lens surgery to insert an intraocular lens into a subject eye is referred to simply as “surgery”. Moreover, in the present specification, for ease of explanation events before surgery is performed are referred to as “prior to surgery”, and events after surgery has been performed are referred to as “following surgery”.

Patent Document 1 discloses an intraocular lens selection device used to select an intraocular lens for insertion into a subject eye. In the intraocular lens selection device disclosed in Patent Document 1, a postoperative residual wavefront aberration of a subject eye is computed as an anticipated value for each intraocular lens model, based on a corneal wavefront aberration of the cornea of the subject eye as obtained by measurement using a measurement means, and a wavefront aberration of the corresponding intraocular lens model. In the intraocular lens selection device disclosed in Patent Document 1, a predetermined postoperative residual wavefront aberration of the subject eye is taken as a target value, and the target value and the anticipated value obtained by the computation are compared for each intraocular lens model in order to identify the intraocular lens model that comes closest to the target value. The intraocular lens selection device disclosed in Patent Document 1 displays information relating to the identified intraocular lens model on a monitor.

### RELATED ART

### Patent Documents

Patent Document 1: Japanese Patent Application Laid-Open (JP-A) No. 2009-34451

### SUMMARY OF INVENTION

An ophthalmic instrument according to a first aspect of the technology disclosed herein includes a light source, an optical system that guides light emitted from the light source onto a retina of a subject eye, a communication section that receives a simulation image generated based on optometry information for the subject eye and intraocular lens information relating to an intraocular lens prescribable for the subject eye, the simulation image corresponding to how vision would appear in a case in which the intraocular lens is prescribed for the subject eye, and a control section that controls the light source and the optical system such that the simulation image received by the communication section is projected onto the retina.

An image generation device according to a second aspect of the technology disclosed herein includes a generation section that generates a simulation image based on optometry information for a subject eye and intraocular lens information relating to an intraocular lens prescribable for the subject eye, the simulation image corresponding to how vision would appear in a case in which the intraocular lens is prescribed for the subject eye, and an output section that outputs the simulation image generated by the generation section to a projection device.

An ophthalmic system according to a third aspect of the technology disclosed herein includes a projection device that projects an image onto a retina of a subject eye, and an image generation device that generates a simulation image based on optometry information for the subject eye and intraocular lens information relating to an intraocular lens prescribable for the subject eye, the simulation image corresponding to how vision would appear in a case in which the intraocular lens is prescribed for the subject eye. The projection device projects the simulation image generated by the image generation device onto the retina.

### BRIEF DESCRIPTION OF DRAWINGS

FIG. 1 is a schematic diagram illustrating an example of an overall configuration of an ophthalmic system according to an exemplary embodiment.

FIG. 2 is a schematic plan view configuration diagram illustrating an example of configuration of a wearable terminal device included in an ophthalmic system according to an exemplary embodiment.

FIG. 3 is a block diagram illustrating an example of a hardware configuration of an electrical system of an ophthalmic system according to an exemplary embodiment.

FIG. 4 is a schematic configuration diagram illustrating an example of configuration of a laser light source included in a wearable terminal device of an ophthalmic system according to an exemplary embodiment.

FIG. 5 is a schematic configuration diagram illustrating an example of configuration of a laser beam splitter included in a wearable terminal device of an ophthalmic system according to an exemplary embodiment.

FIG. 6 is a flowchart illustrating an example of a flow of server-side processing according to an exemplary embodiment.

FIG. 7 is a flowchart illustrating an example of a flow of postoperative field-of-view simulation processing included in server-side processing according to an exemplary embodiment.

FIG. 8 is a flowchart illustrating an example of a flow of terminal-side processing according to an exemplary embodiment.

FIG. 9 is a schematic screen diagram illustrating an example of a screen displayed on a display as a result of executing server-side processing according to an exemplary embodiment.

FIG. 10 is a functional block diagram illustrating an example of relevant functionality of a server device according to an exemplary embodiment.

FIG. 11 is a functional block diagram illustrating an example of relevant functionality of a wearable terminal device according to an exemplary embodiment.

FIG. 12 is a schematic diagram illustrating a modified example of a wearable terminal device according to an exemplary embodiment.

FIG. 13 is a schematic diagram illustrating a first modified example of an ophthalmic system according to an exemplary embodiment.

FIG. 14 is a schematic diagram illustrating an example of a manner in which a terminal-side program according to an exemplary embodiment is installed on a wearable terminal device.

FIG. 15 is a schematic diagram illustrating an example of a manner in which a server-side program according to an exemplary embodiment is installed on a server device.

FIG. 16 is a schematic diagram illustrating a second modified example of an ophthalmic system according to an exemplary embodiment.

### DESCRIPTION OF EMBODIMENTS

Explanation follows regarding an example of an exemplary embodiment according to the technology disclosed herein, with reference to the appended drawings.

First, explanation will be given regarding the meaning of the terms employed in the following description.

In the following description MEMS is employed as an abbreviation of micro electro mechanical systems. In the following description I/F is employed as an abbreviation of “interface”. In the following description I/O is employed as an abbreviation of “input/output interface”. In the following description USB is employed as an abbreviation of “universal serial bus”. In the following description ID is employed as an abbreviation of “identification”.

In the following description CPU is employed as an abbreviation of “central processing unit”. In the following description RAM is employed as an abbreviation of “random access memory”. In the following description HDD is employed as an abbreviation of “hard disk drive”. In the following description EEPROM is employed as an abbreviation of “electrically erasable and programmable read only memory”. In the following description SSD is employed as an abbreviation of “solid state drive”. In the following description DVD-ROM is employed as an abbreviation of “digital versatile disk read only memory”.

In the following description ASIC is employed as an abbreviation of “application specific integrated circuit”. In the following description FPGA is employed as an abbreviation of “field programmable gate array”.

Moreover, in the present exemplary embodiments, a left-right direction indicates, for example, the direction of a straight line passing through the center of the pupil of the right eye of a patient and through the center of the pupil of the left eye of the patient. Note that in the following, for ease of explanation, the “left-right direction” is referred to as the “X direction”, a direction from the center of the pupil of a subject eye toward the rear pole of the subject eye is referred to as the “Z direction”, and a direction perpendicular to both the X direction and the Z direction is referred to as the “Y direction”.

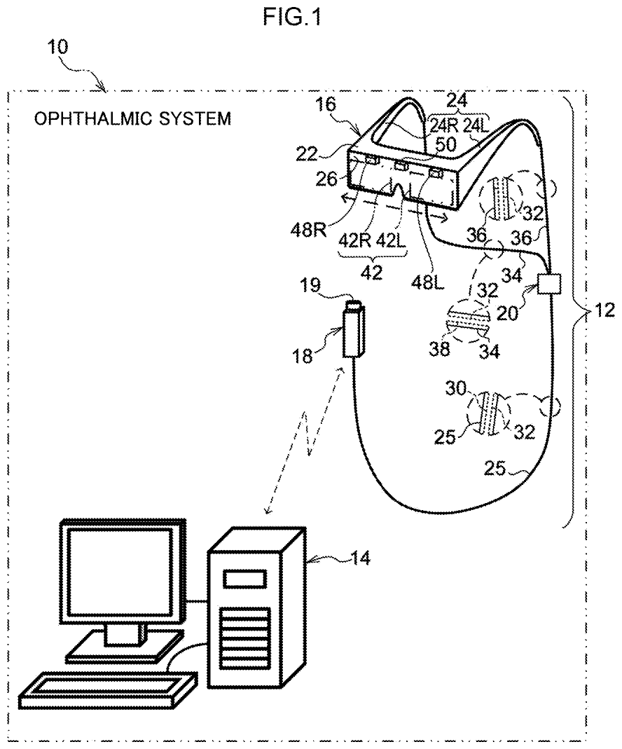

As illustrated in FIG. 1 as an example, an ophthalmic system 10 is a system employed to project pictures, encompassing still images and moving images, onto the retina of a subject eye of a patient prior to the patient undergoing surgery, in order to allow the patient to understand how their vision will appear following surgery. The ophthalmic system 10 includes a wearable terminal device 12 as an example of a projection device and an ophthalmic instrument according to the technology disclosed herein, and a server device 14 serving as an example of an image generation device according to the technology disclosed herein.

The wearable terminal device 12 includes an eyewear terminal device 16, a control device 18, and a laser beam splitter 20.

The eyewear terminal device 16 is one sort of glasses-type terminal device worn by a patient. Reference here to “patient” indicates a cataract patient for whom removal of the crystalline lens from the subject eye followed by insertion of an intraocular lens is planned.

Although an example is described in which the patient is a cataract sufferer in a first exemplary embodiment, the technology disclosed herein is not limited thereto and, for example, the patient may be undergoing corrective treatment for myopia. In such cases, an intraocular lens is inserted the subject eye of the patient without removing the crystalline lens.

Similarly to ordinary glasses, the eyewear terminal device 16 includes a rim piece 22 and a temple piece 24. The eyewear terminal device 16 also includes a patient projection section 26.

The rim piece 22 holds the patient projection section 26 in front of the eyes of the patient. The temple piece 24 is broadly divided into a left temple piece 24L and a right temple piece 24R. One end portion of the left temple piece 24L is attached to a left end portion of the rim piece 22, and the right temple piece 24R is attached to the right end portion of the rim piece 22. The left temple piece 24L is hooked over the left ear of the patient, and the right temple piece 24R is hooked over the right ear of the patient.

The control device 18 is, for example, employed by being grasped by the patient, or is worn on the clothes or on the body of the patient. The control device 18 is equipped with a response button 19. The response button 19 is pressed by the patient when the patient responds to questioning by a medical service professional. A “medical service professional” as referred to here is a person providing a medical service to the patient by presenting pictures to the patient using the ophthalmic system 10. A doctor is one example of a medical service professional.

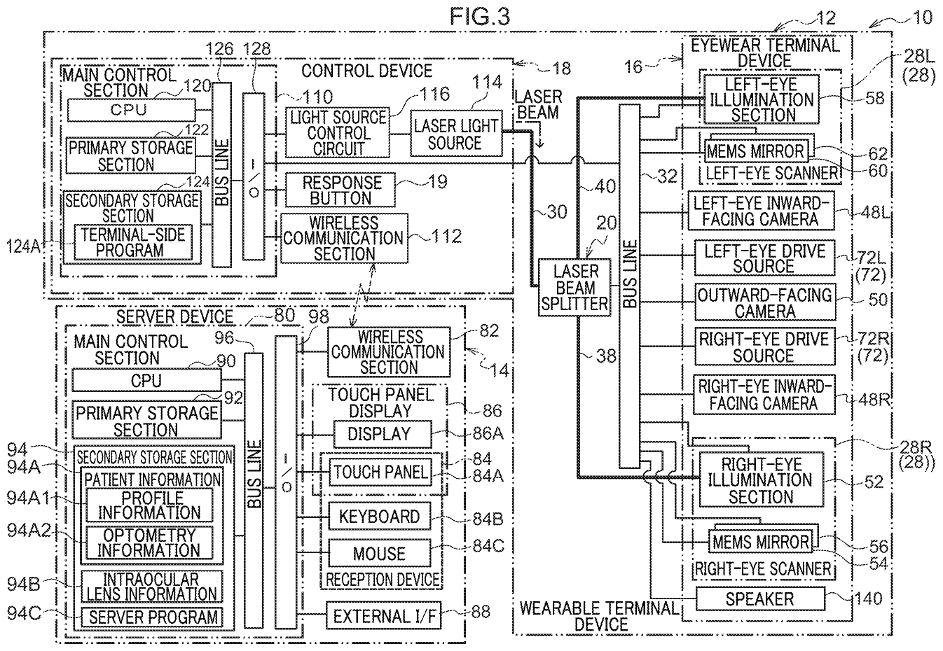

The control device 18 is connected to the server device 14 so as to be capable of wireless communication therewith through a wireless communication section 112 (see FIG. 3), described later, and the control device 18 exchanges various kinds of information with the server device 14. The control device 18 is connected to the laser beam splitter 20 via a cable 25 and controls the laser beam splitter 20.

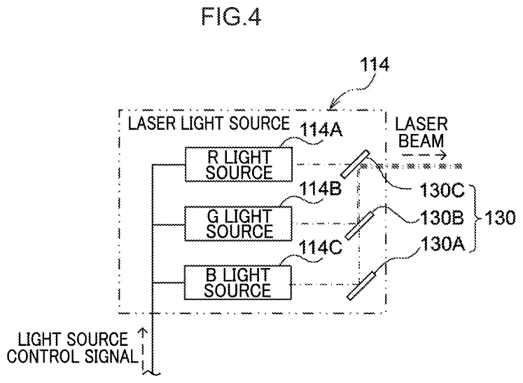

The cable 25 includes an optical fiber 30 and a bus line 32. The control device 18 includes a laser light source 114 (see FIG. 4) that emits a laser beam, and controls the laser light source 114 so as to supply a laser beam to the laser beam splitter 20 through the optical fiber 30. The control device 18 also controls the laser beam splitter 20 through the bus line 32.

The laser beam splitter 20 is connected to the eyewear terminal device 16 via cables 34, 36. The cable 34 is connected to the right temple piece 24R, and the cable 36 is connected to the left temple piece 24L. The cables 34, 36 both include the bus line 32. Thus the control device 18 exchanges various kinds of electrical signal with the eyewear terminal device 16 through the bus line 32.

The cable 34 includes an optical fiber 38, and the cable 36 includes an optical fiber 40. The laser beam splitter 20 splits the laser beam supplied from the control device 18 through the optical fiber 30 selectively between the optical fiber 38 and the optical fiber 40. One of the laser beams obtained by splitting with the laser beam splitter 20 is supplied into the eyewear terminal device 16 through the optical fiber 38. The other of the laser beams obtained by splitting with the laser beam splitter 20 is supplied into the eyewear terminal device 16 through the optical fiber 40.

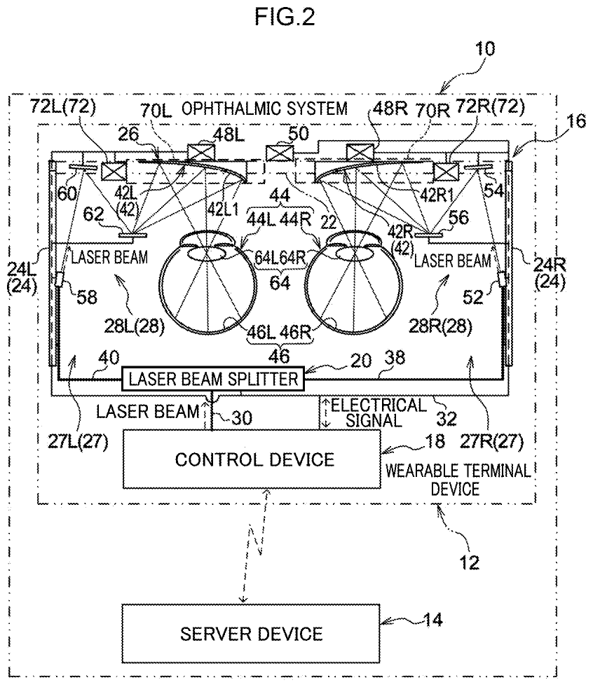

The patient projection section 26 includes reflection mirrors 42. The reflection mirrors 42 are an example of a reflection member according to the technology disclosed herein. The reflection mirrors 42 guide the laser beam onto the retina 46 of the subject eye 44 of the patient by reflecting the laser beam supplied from the laser beam splitter 20 through the cables 34, 36, as illustrated for example in FIG. 2. Note that the subject eyes 44 include a right eye 44R and a left eye 44L, as illustrated for example in FIG. 2.

The reflection mirrors 42 are broadly composed of a right-eye reflection mirror 42R and a left-eye reflection mirror 42L. The right-eye reflection mirror 42R is held by the rim piece 22 so as to be positioned in front of the right eye 44R of the patient when the eyewear terminal device 16 is in a correctly worn state. The left-eye reflection mirror 42L is held by the rim piece 22 so as to be positioned in front of the left eye 44L of the patient when the eyewear terminal device 16 is in a correctly worn state.

The right-eye reflection mirror 42R guides a laser beam onto the retina 46R of the right eye 44R of the patient by reflecting the laser beam supplied from the laser beam splitter 20 through the optical fiber 38, as illustrated for example in FIG. 2. The left-eye reflection mirror 42L guides a laser beam onto the retina 46L of the left eye 44L of the patient by reflecting the laser beam supplied from the laser beam splitter 20 through the optical fiber 40, as illustrated for example in FIG. 2.

The eyewear terminal device 16 is equipped with a right-eye inward-facing camera 48R, a left-eye inward-facing camera 48L, and an outward-facing camera 50. The right-eye inward-facing camera 48R, the left-eye inward-facing camera 48L, and the outward-facing camera 50 image an imaging subject under control from the control device 18.

The right-eye inward-facing camera 48R, the left-eye inward-facing camera 48L, and the outward-facing camera 50 are attached to an upper edge of the rim piece 22. The right-eye inward-facing camera 48R is provided at a position shifted away from the right-eye reflection mirror 42R in the Y direction, and images the anterior segment of the right eye 44R as an imaging subject from diagonally above a region in front of the right eye 44R. The left-eye inward-facing camera 48L is provided at a position shifted away from the left-eye reflection mirror 42L in the Y direction, and images the anterior segment of the left eye 44L as an imaging subject from diagonally above a region in front of the left eye 44L. The right-eye inward-facing camera 48R and the left-eye inward-facing camera 48L are examples of anterior segment cameras according to the technology disclosed herein.

The outward-facing camera 50 is attached to a central portion of the upper edge of the rim piece 22, for example so as to be positioned in front of the patient between the eyebrows in a state in which the eyewear terminal device 16 is being worn. The outward-facing camera 50 images an external field of vision. Namely, the outward-facing camera 50 images as an imaging subject a region in front of the patient as if seen through the patient projection section 26 from the perspective of the patient in a state in which the eyewear terminal device 16 is being worn. The “region in front” referred to herein is, for example, the field of vision of the patient as it would appear to the patient in a case in which they not wearing the eyewear terminal device 16, namely a region in actual space in the direction of gaze of the patient. Note that the outward-facing camera 50 is an example of a field of vision camera according to the technology disclosed herein.

The server device 14 generates pictures, including still images and moving images, and transmits the generated pictures to the control device 18. The control device 18 receives the pictures transmitted from the server device 14 and supplies laser beams to the eyewear terminal device 16 through the optical fibers 30, 38, 40 in response to the received pictures.

Note that although explanation has been given in the present exemplary embodiment of an example in which wireless communication is performed between the wearable terminal device 12 and the server device 14, the technology disclosed herein is not limited thereto. For example, wired communication may be performed between the wearable terminal device 12 and the server device 14.

As illustrated as an example in FIG. 2, the eyewear terminal device 16 includes an optical system 27. The optical system 27 guides the laser beams onto the retina 46. The optical system 27 includes a scanner 28 and the reflection mirrors 42. The scanner 28 scans laser beams supplied from the control device 18 through the laser beam splitter 20. The reflection mirrors 42 reflect the laser beams being scanned by the scanner 28 onto the retinas 46.

The optical system 27 includes a right-eye optical system 27R and a left-eye optical system 27L. The laser beam splitter 20 splits the laser beam supplied from the control device 18 through the optical fiber 30 in a laser beam for the right-eye optical system 27R and a laser beam for the left-eye optical system 27L.

A laser beam that has passed from the laser beam splitter 20, through the optical fiber 38, and been shone from a right-eye illumination section 52 is guided onto the retina 46R by the right-eye optical system 27R. A laser beam that has passed from the laser beam splitter 20, through the optical fiber 40, and been supplied from a left-eye illumination section 58 is guided onto the retina 46L by the left-eye optical system 27L.

The scanner 28 includes a right-eye scanner 28R and a left-eye scanner 28L. The right-eye optical system 27R includes the right-eye scanner 28R and the right-eye reflection mirror 42R. The left-eye optical system 27L includes the left-eye scanner 28L and the left-eye reflection mirror 42L.

The right-eye scanner 28R includes MEMS mirrors 54, 56, and scans the laser beam supplied through the right-eye illumination section 52. The right-eye illumination section 52 shines the laser beam supplied from the laser beam splitter 20 through the optical fiber 38. The MEMS mirror 54 is disposed on the direction the laser beam is shone in by the right-eye illumination section 52, and the MEMS mirror 54 reflects the laser beam being shone from the right-eye illumination section 52 so as to be guided onto the MEMS mirror 56. The MEMS mirror 56 reflects the laser beam guided by the MEMS mirror 54 so as to be guided onto the right-eye reflection mirror 42R.

For example, the MEMS mirror 54 scans the laser beam in the Y direction, and the MEMS mirror 56 scans the laser beam in the X direction. Two-dimensional scanning on the retina is enabled by the MEMS mirrors 54, 56, enabling a picture to be scanned and projected onto the retina in two dimensions.

Obviously a configuration may be adopted in which the MEMS mirror 54 scans in the X direction and the MEMS mirror 56 scans in the Y direction.

Furthermore, the right-eye scanner 28R may be configured by employing the reflection mirror 42R and a MEMS mirror 56 capable of scanning in the XY directions.

The right-eye reflection mirror 42R reflects the laser beam scanned by the right-eye scanner 28R onto the retina 46R.

The right-eye reflection mirror 42R includes a curved surface 42R1. The curved surface 42R1 is a surface formed so as to be concave as viewed from the right eye 44R of the patient in a state in which the eyewear terminal device 16 is being worn. Due to the laser beam guided by the MEMS mirror 56 being reflected at the curved surface 42R1, the laser beam is guided through a crystalline lens 64R behind the pupil of the right eye 44R and onto the retina 46R of the right eye 44R.

The left-eye scanner 28L includes MEMS mirrors 60, 62, and scans the laser beam supplied through the left-eye illumination section 58. The left-eye illumination section 58 shines the laser beam supplied from the laser beam splitter 20 through the optical fiber 40. The MEMS mirror 60 is disposed on the direction of illumination of the laser beam by the left-eye illumination section 58, and the MEMS mirror 60 reflects the laser beam shone from the left-eye illumination section 58 so as to be guided onto the MEMS mirror 62. The MEMS mirror 62 reflects the laser beam guided by the MEMS mirror 60 so as to be guided onto the left-eye reflection mirror 42L.

For example, the MEMS mirror 60 scans the laser beam in the Y direction, and the MEMS mirror 62 scans the laser beam in the X direction. Two-dimensional scanning on the retina is enabled by the MEMS mirrors 60, 62, enabling a picture to be scanned and projected onto the retina in two dimensions.

Obviously a configuration may be adopted in which the MEMS mirror 60 scans in the X direction and the MEMS mirror 62 scans in the Y direction.

Furthermore, the left-eye scanner 28L may be configured by employing the reflection mirror 42L and a MEMS mirror 56 capable of scanning in the XY directions.

Although the MEMS mirrors 54, 56, 60, 62 are given as examples in the example illustrated in FIG. 2, the technology disclosed herein is not limited thereto. For example, instead of the MEMS mirrors 54, 56, 60, 62, or together with one or more of the MEMS mirrors 54, 56, 60, 62, a mirror such as a galvanometer mirror and/or a polygon mirror or the like that enables electrical control of the position on the reflection face may be employed.

The left-eye reflection mirror 42L reflects the laser beam scanned by the left-eye scanner 28L onto the retina 46L.

The left-eye reflection mirror 42L includes a curved surface 42L1. The curved surface 42L1 is a surface formed so as to be concave as viewed from the left eye 44L of the patient in a state in which the eyewear terminal device 16 is being worn. Due to the laser beam guided by the MEMS mirror 62 being reflected at the curved surface 42L1, the laser beam is guided through a crystalline lens 64L behind the pupil of the left eye 46R and onto the retina 46L of the left eye 44L.

Note that when there is no need to discriminate between the crystalline lenses 64R, 64L in the description below, for ease of explanation they will be referred to as “crystalline lenses 64”.

The patient projection section 26 further includes a right-eye sliding mechanism 70R, a left-eye sliding mechanism 70L, a right-eye drive source 72R, and a left-eye drive source 72L. Examples of the right-eye drive source 72R and the left-eye drive source 72L include a stepping motor, a solenoid, and a piezoelectric element or the like. Note that when there is no need to discriminate between the right-eye drive source 72R and the left-eye drive source 72L in the description below, for ease of explanation they will be referred to as “mirror drive sources 72”.

The right-eye sliding mechanism 70R is attached to the rim piece 22, and is held thereby so as to enable the right-eye reflection mirror 42R to slide in the left-right direction. The right-eye sliding mechanism 70R is connected to the right-eye drive source 72R, and slides the right-eye reflection mirror 42R in the left-right direction on receipt of motive force generated by the right-eye drive source 72R.

The left-eye sliding mechanism 70L is attached to the rim piece 22, and is held thereby so as to enable the left-eye reflection mirror 42L to slide in the left-right direction. The left-eye sliding mechanism 70L is connected to the left-eye drive source 72L, and slides the left-eye reflection mirror 42L in the left-right direction on receipt of motive force generated by the left-eye drive source 72L.

In the ophthalmic system 10 according to the present exemplary embodiment, a picture based on the laser beam is projected onto the retina 46 of the subject eye 44 by a Maxwellian view optical system. Reference here to “Maxwellian view optical system” indicates an optical system in which laser beams are converged by the crystalline lenses 64 behind the pupils of the subject eyes 44, and a picture arising from the laser beam is projected onto the retina 46 of the subject eye 44 by the laser beams converged by the crystalline lens 64 being shone onto the retina 46 of the subject eye 44. In the ophthalmic system 10 according to the present exemplary embodiment, the Maxwellian view optical system is implemented by the scanner 28 and the mirror drive sources 72 being controlled by the control device 18.

As illustrated for example in FIG. 3, the server device 14 includes a main control section 80, a wireless communication section 82, a reception device 84, a touch panel display 86, and an external I/F 88. Note that the main control section 80 is an example of a computer according to the technology disclosed herein.

The main control section 80 includes a CPU 90, a primary storage section 92, a secondary storage section 94, a bus line 96, and an I/O 98. The CPU 90, the primary storage section 92, and the secondary storage section 94 are connected together through the bus line 96. The I/O 98 is connected to the bus line 96.

The CPU 90 controls the server device 14 overall. The primary storage section 92 is volatile memory employed as a work area or the like when various programs are being executed. An example of the primary storage section 92 is RAM. The secondary storage section 94 is non-volatile memory to store a program and various parameters and the like employed to control the basic operation of the server device 14. Examples of the secondary storage section 94 include a HDD, EEPROM, and flash memory or the like.

The wireless communication section 82 is connected to the I/O 98. The CPU 90 outputs to the wireless communication section 82 an electrical signal for transmission to the control device 18. The wireless communication section 82 transmits the electrical signal input from the CPU 90 to the control device 18 using radio waves. The wireless communication section 82 also receives radio waves from the control device 18, and outputs to the CPU 90 an electrical signal according to the received radio waves.

The reception device 84 is an example of a reception section of the technology disclosed herein. The reception device 84 includes a touch panel 84A, a keyboard 84B, and a mouse 84C, with the touch panel 84A, the keyboard 84B, and the mouse 84C being connected to the I/O 98. This accordingly enables the CPU 90 to ascertain various instructions received by each of the touch panel 84A, the keyboard 84B, and the mouse 84C.

The external I/F 88 is connected to external devices, such as a personal computer, and/or a USB memory or the like, and is employed to exchange various information between the external devices and the CPU 90.

The touch panel display 86 includes a display 86A and a touch panel 84A. The display 86A is an example of a display section according to the technology disclosed herein. The display 86A is connected to the I/O 98 and displays various information including pictures under control from the CPU 90. The touch panel 84A is a transparent touch panel superimposed on the display 86A.

The secondary storage section 94 stores patient information 94A, intraocular lens information 94B, and a server-side program 94C.

The patient information 94A is information related to the patient. In the present exemplary embodiment, the patient information 94A includes patient profile information 94A1 (for example, an ID to identify the patient, patient name, patient gender, patient age, physical information, past treatment history, current patient information such as hospitalization status, risk of disease, and physical state and the like) and optometry information 94A2 relating to optometry performed on the patient. The optometry information 94A2 includes other information related to the left eye/right eye of the patient (for example, corneal refractive power, corneal wavefront aberration, visual acuity, myopia/hyperopia/astigmatism, field of view, eye axial length, fundus photographs or the like that is information obtained with a different ophthalmic instrument). Examples of the different ophthalmic instrument include a refractive power measurement instrument, eye axial length measurement instrument, a visual acuity detector, an anterior segment measurement instrument, a posterior segment measurement instrument, and the like. The optometry information 94A2 is an example of “optometry information for the subject eye” according to the technology disclosed herein. The optometry information may be stored on a non-illustrated ophthalmic server and acquired from the ophthalmic server by the server device 14 through the wireless communication section 82 or the external I/F 88.

The intraocular lens information 94B is information relating to an intraocular lens prescribed for the subject eye 44, and may alternatively be described as information expressing characteristics of an intraocular lens due to be inserted into the subject eye 44. The intraocular lens information 94B is stored in the secondary storage section 94 for each different intraocular lens. The intraocular lens information 94B is an example of intraocular lens information according to the technology disclosed herein.

In the present exemplary embodiment, the intraocular lens information 94B includes information regarding the model, manufacturer, A constant, an anticipated postoperative anterior chamber depth (ACD; in mm), spherical aberration (SA; in μm) (corrected spherical aberration) of the intraocular lens, whether or not the intraocular lens is a colored lens, and the lens material. SA refers to the corrected spherical aberration in a case in which the intraocular lens is inserted into the subject eye 44. Note that the SA is calculated in various manners by different manufacturers. Since these are already widely known, explanation thereof is omitted herein.

The server-side program 94C is an example of a program according to the technology disclosed herein. The CPU 90 reads the server-side program 94C from the secondary storage section 94 and expands the read server-side program 94C into the primary storage section 92. The CPU 90 then executes the server-side program 94C that has been expanded into the primary storage section 92.

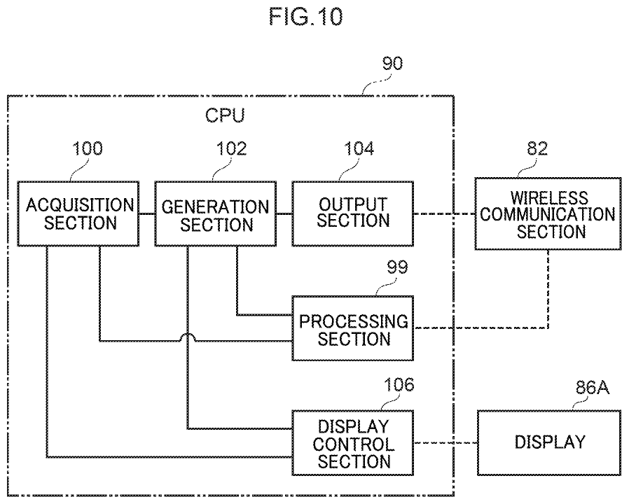

As illustrated in the example of FIG. 10, the CPU 90 operates as a processing section 99, an acquisition section 100, a generation section 102, an output section 104, and a display control section 106 by executing the server-side program 94C.

The processing section 99 performs processing required for the CPU 90 to operate as the acquisition section 100, the generation section 102, the output section 104, and the display control section 106. The acquisition section 100 acquires an original picture from plural original pictures of different scenes in response to an instruction received by the reception device 84. The generation section 102 generates a simulation picture by performing conversion or image processing on the original picture acquired by the acquisition section 100, based on the optometry information 94A2 and the intraocular lens information 94B. The output section 104 outputs the simulation picture generated by the generation section 102 to the wearable terminal device 12 by performing wireless communication with the wearable terminal device 12 through a wireless communication section 83.

The display control section 106 controls the display 86A such that the original picture and the simulation picture are displayed on the display 86A. Note that the original picture is an example of an original image according to the technology disclosed herein.

Note that in the present exemplary embodiment, an example is given in which the CPU 90 operates as the processing section 99, the acquisition section 100, the generation section 102, the output section 104, and the display control section 106. However, the technology disclosed herein is not limited thereto. For example, distributed processing may be performed using a main CPU and plural processors such as image processing processors. For example, configuration may be made such that the main CPU operates as the processing section 99 and the acquisition section 100, and the image processing processors operate as the generation section 102, the output section 104, and the display control section 106.

As illustrated in FIG. 3 as an example, as well as the response button 19 mentioned above the control device 18 is equipped with a main control section 110, the wireless communication section 112, the laser light source 114, and a light source control circuit 116. The main control section 110 is an example of a computer according to the technology disclosed herein.

The main control section 110 includes a CPU 120, a primary storage section 122, a secondary storage section 124, a bus line 126, and an I/O 128. The CPU 120, the primary storage section 122, and the secondary storage section 124 are connected together through the bus line 126. The I/O 128 is connected to the bus line 126.

The CPU 120 controls the wearable terminal device 12 overall. The primary storage section 122 is volatile memory employed as a work area or the like when various programs are being executed. An example of the primary storage section 122 is RAM. The secondary storage section 124 is non-volatile memory to store a program and various parameters and the like employed to control the basic operation of the wearable terminal device 12. Examples of the secondary storage section 124 include a HDD, EEPROM, and flash memory or the like.

The response button 19 is connected to the I/O 128, and a response signal is output from the response button 19 to the CPU 120 when the response button 19 is pressed.

The wireless communication section 112 is an example of a communication section of technology disclosed herein, and is connected to the I/O 128. The CPU 120 outputs to the wireless communication section 112 an electrical signal for transmission to the server device 14. The wireless communication section 112 transmits the electrical signal input from the CPU 120 to the server device 14 using radio waves. The wireless communication section 112 also receives radio waves from the server device 14, and outputs to the CPU 120 an electrical signal according to the received radio waves.

The laser light source 114 is connected to the laser beam splitter 20 through the optical fiber 30. The laser light source 114 generates a laser beam, and the generated laser beam is emitted into the laser beam splitter 20 through the optical fiber 30.

The laser light source 114 is connected to the light source control circuit 116. The light source control circuit 116 is connected to the I/O 128. The light source control circuit 116 supplies light source control signals to the laser light source under instruction from the CPU 120, and controls the laser light source 114.

As illustrated in the example in FIG. 4, the laser light source 114 includes a R light source 114A, a G light source 114B, a B light source 114C, and a mirror unit 130.

The R light source 114A emits an R laser beam that is an R laser beam from R (red), G (green), and B (blue). The G light source 114B emits a G laser beam that is a G laser beam from R, G, and B. The B light source 114C emits a B laser beam that is a B laser beam from out R, G, and B.

The mirror unit 130 is equipped with a first mirror 130A, a second mirror 130B, and a third mirror 130C. From the first mirror 130A, the second mirror 130B, and the third mirror 130C, the second mirror 130B is a dichroic mirror that transmits the B laser beam while reflecting the G laser beam. The third mirror 130C is also a dichroic mirror, and transmits the R laser beam while reflecting the G laser beam and the B laser beam.

The first mirror 130A is disposed in the direction in which the B laser beam is emitted by the B light source 114C, and guides the B laser beam to the second mirror 130B by reflecting the B laser beam emitted from the B light source 114C.

The second mirror 130B is disposed in the direction in which the G laser beam is emitted by the G light source 114B and is also on the direction of progression of the B laser beam reflected by the first mirror 130A. The second mirror 130B guides the G laser beam to the first mirror 130A by reflecting the G laser beam emitted from the G light source 114B, and also guides the B laser beam to the first mirror 130A by transmitting the B laser beam reflected by the first mirror 130A.

The third mirror 130C is disposed in the direction in which the R laser beam is emitted by the R light source 114A and also on the direction of progression of the G laser beam reflected by the second mirror 130B as well as on the direction of progression of the G laser beam transmitted through the second mirror 130B. The third mirror 130C transmits the R laser beam emitted from the R light source 114A. The third mirror 130C externally emits the R laser beam, the G laser beam, and the B laser beam by reflecting the G laser beam and the B laser beam onto the same direction as that of the R laser beam. In the following explanation, for ease of explanation the R laser beam, the G laser beam, and the B laser beam emitted externally from the laser light source 114 are simply referred to as the “laser beam”.

As illustrated for example in FIG. 3, the bus line 32 is connected to the I/O 128, and the laser beam splitter 20 is connected to the bus line 32. Thus the laser beam splitter 20 operates under control from the CPU 120.

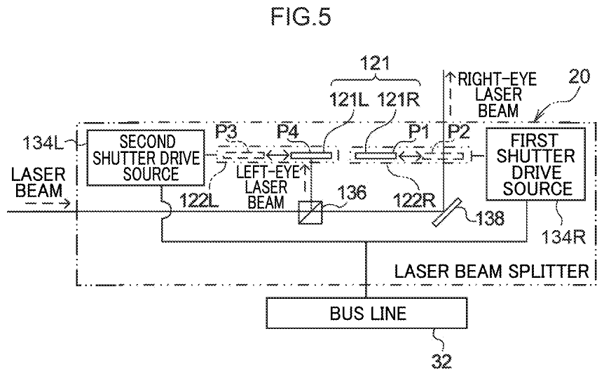

In the example illustrated in FIG. 5, the laser beam splitter 20 includes a right-eye shutter 121R, a left-eye shutter 121L, a first sliding mechanism 122R, a second sliding mechanism 122L, a first shutter drive source 134R, a second shutter drive source 134L, a beam splitter 136, and a reflection mirror 138.

When there is no need to discriminate between the right-eye shutter 121R and the left-eye shutter 121L in the description below, for ease of explanation they will be referred to as “shutters 121”.

The beam splitter 136 reflects and transmits the laser beam supplied from the laser light source 114 through the optical fiber 130. A left-eye laser beam that is a laser beam reflected by the beam splitter 136 proceeds toward the inlet port of the optical fiber 40 (see FIG. 1 and FIG. 2).

The reflection mirror 138 reflects the laser beam transmitted through the beam splitter 136. A right-eye laser beam that is a laser beam reflected by the reflection mirror 138 proceeds toward the inlet port of the optical fiber 38 (see FIG. 1 and FIG. 2).

The first sliding mechanism 122R holds the right-eye shutter 121R so as to be capable of sliding between a first position P1 and a second position P2. The first position P1 indicates a position where the right-eye laser beam is transmitted and guided into the inlet port of the optical fiber 38, and the second position P2 indicates a position where the right-eye laser beam is blocked.

The second sliding mechanism 122L holds the left-eye shutter 121L so as to be capable of sliding between a third position P3 and a fourth position P4. The third position P3 indicates a position where the left-eye laser beam is transmitted and guided into the inlet port of the optical fiber 40, and the fourth position P4 indicates a position where the left-eye laser beam is blocked.

Examples of the first shutter drive source 134R and the second shutter drive source 134L include stepping motors, solenoids, and piezoelectric elements or the like. The first shutter drive source 134R and the second shutter drive source 134L are connected to the bus line 32, and the first shutter drive source 134R and the second shutter drive source 134L operate under control from the CPU 120.

The first sliding mechanism 122R is connected to the first shutter drive source 134R, and slides the right-eye shutter 121R between the first position P1 and the second position P2 on receipt of motive force generated by the first shutter drive source 134R.

The second sliding mechanism 122L is connected to the second shutter drive source 134L and slides the left-eye shutter 121L between the third position P3 and the fourth position P4 on receipt of motive force generated by the second shutter drive source 134L.

In the example illustrated in FIG. 5, the right-eye laser beam is supplied into the optical fiber 38 by the right-eye shutter 121R being disposed at the first position P1, and the left-eye laser beam is blocked by the left-eye shutter 121L by the left-eye shutter 121L being disposed at the fourth position P4. Note that although the right-eye shutter 121R and the left-eye shutter 121L are mechanical shutters configured by mechanisms in the present exemplary embodiment, the technology disclosed herein is not limited thereto. For example, shutters employing liquid crystal technology or the like to block light electrically may be employed instead of the right-eye shutter 121R and the left-eye shutter 121L.

As illustrated in FIG. 3 as an example, the eyewear terminal device 16 is provided with a speaker 140. The speaker 140 is provided on the temple piece 24. The speaker 140 is connected to the bus line 32, and outputs audio under control from the CPU 120. The speaker 140 may be a speaker that directly imparts a sound wave to the eardrum of the patient, or may be a bone conduction speaker that indirectly transmits vibrations to the eardrum of the patient.

The right-eye drive source 72R and the left-eye drive source 72L are connected to the bus line 32, and the CPU 120 controls the right-eye drive source 72R and the left-eye drive source 72L.

The right-eye inward-facing camera 48R, the left-eye inward-facing camera 48L, and the outward-facing camera 50 are connected to the bus line 32, and the CPU 120 exchanges various kinds of information with the right-eye inward-facing camera 48R, the left-eye inward-facing camera 48L, and the outward-facing camera 50.

The right-eye illumination section 52, the left-eye illumination section 58, and the MEMS mirrors 54, 56, 60, 62 are also connected to the bus line 32, and the CPU 120 controls the right-eye illumination section 52, the left-eye illumination section 58, and the MEMS mirrors 54, 56, 60, 62.

The secondary storage section 124 stores a terminal-side program 124A. The terminal-side program 124A is an example of a program according to the technology disclosed herein. The CPU 120 reads the terminal-side program 124A from the secondary storage section 124, and expands the read terminal-side program 124A into the primary storage section 122. The CPU 120 executes the terminal-side program 124A that has been expanded into the primary storage section 122.

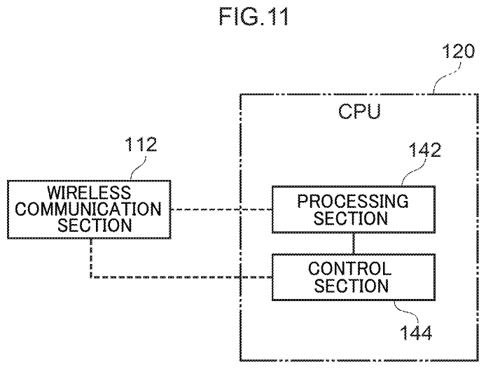

As illustrated in FIG. 11, for example, the CPU 120 operates as a processing section 142 and a control section 144 by executing the terminal-side program 124A. The processing section 142 performs processing required for the CPU 120 to operate as the control section 144. The processing section 142 controls the wireless communication section 112 such that the wireless communication section 112 receives the simulation picture transmitted from the server device 14. The control section 144 also controls the laser light source 114 and the optical system 27 so as to project the simulation picture received by the wireless communication section 112 onto the retina 46. The control section 144 also controls the wireless communication section 112 so as to transmit an image obtained by imaging with the outward-facing camera 50 to the server device 14.

Although an example in which the CPU 120 operates as the processing section 142 and the control section 144 is described in the present exemplary embodiment, the technology disclosed herein is not limited thereto. For example, distributed processing may be performed using a main CPU and plural processors such as sub-CPUs. For example, configuration may be made in which the main CPU operates as the processing section 142, and the sub-CPUs operate as the control section 144.

Explanation next follows regarding operation of the sections of the ophthalmic system 10 according to the technology disclosed herein.

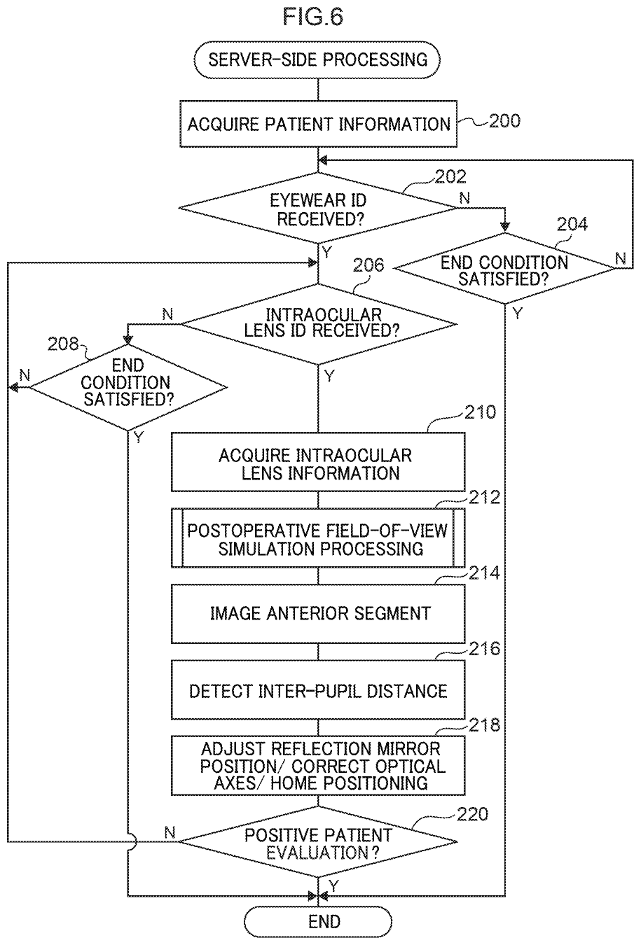

First explanation will be given regarding server-side processing implemented by the CPU 90 executing the server-side program 94C when an instruction to start executing of server-side processing is received by the reception device 84, with reference to FIG. 6. Note that the server-side processing illustrated in FIG. 6 is processing performed for a patient prior to surgery in order for the patient to confirm how their vision will appear following surgery.

For ease of explanation, the following description assumes that a patient is appropriately wearing the wearable terminal device 12.

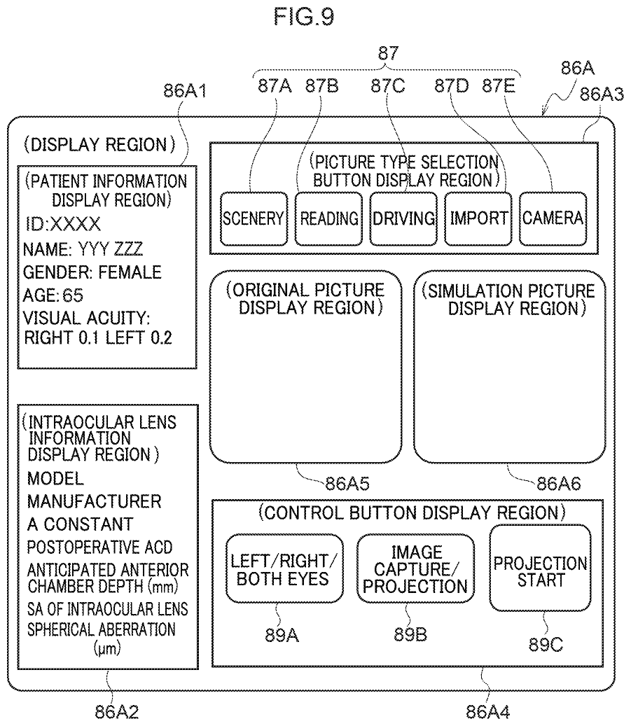

For ease of explanation, the following description also assumes that a screen including plural display regions is being displayed on the display 86A, as illustrated in FIG. 9 as an example. The plural display regions referred to here indicate a patient information display region 86A1, an intraocular lens information display region 86A2, a picture type selection button display region 86A3, a control button display region 86A4, an original picture display region 86A5, and a simulation picture display region 86A6.

For ease of explanation, the following description assumes that the patient information display region 86A1, the intraocular lens information display region 86A2, the original picture display region 86A5, and the simulation picture display region 86A6 are in a non-displayed state when the server-side processing starts.

For ease of explanation, the following description assumes that the picture type selection button display region 86A3 is already displaying a scenery button 87A, a reading button 87B, a driving button 87C, an import button 87D, and a camera button 87E, as illustrated in FIG. 9 as an example.

The scenery button 87A is a software key pressed to cause the CPU 90 to acquire a picture representing scenery as the original picture. The reading button 87B is a software key pressed to cause the CPU 90 to acquire a picture including a large amount of typed text such as in a typical paperback book or newspaper as the original picture. The driving button 87C is a software key pressed to cause the CPU 90 to acquire a picture representing scenery as viewed from a driving seat when driving a car as the original picture. The import button 87D is a software key pressed to cause the CPU 90 to acquire a picture from an external device through the external I/F 88 as the original picture. The camera button 87E is a software key pressed to cause the CPU 90 to operate the outward-facing camera 50 and to cause the CPU 90 to acquire a picture obtained by imaging with the outward-facing camera 50 as the original picture.

For ease of explanation, in the following description an original picture that is a picture representing scenery is referred to as a “scenery original picture”. Moreover, for ease of explanation, in the following description an original picture that is a picture including a large amount of typed text such as in a typical paperback book or newspaper is referred to as a “reading original picture”. For ease of explanation, in the following description an original picture that is a picture representing scenery as viewed from a driving seat when driving a car is referred to as a “driving original picture”. For ease of explanation, in the following description an original picture that is a picture acquired by the CPU 90 from an external device through the external I/F 88 is referred to as an “imported original picture”. For ease of explanation, in the following description an original picture that is a picture obtained by imaging with the outward-facing camera 50 is referred to as a “camera original picture”.

The camera original picture is an example of a field of vision image according to the technology disclosed herein. The camera original picture is transmitted to the server device 14 by the processing section 142 under control from the control section 144.

Moreover, for ease of explanation, the following description assumes that a scenery original picture, a reading original picture, and a driving original picture are pre-stored in the secondary storage section 94.

For ease of explanation, the following description assumes that an external device is connected to the external I/F 88 and that the CPU 90 is in a state capable of acquiring an imported original picture from the external device through the external I/F 88.

For ease of explanation, in the following description, when there is no need to discriminate between the scenery button 87A, the reading button 87B, the driving button 87C, the import button 87D, and the camera button 87E, they will be referred to as “picture selection buttons 87” with their individual reference numerals omitted. Note that there is no limitation to scenes including scenery, reading, driving, and the like, and action pictures, for example of sports, and/or pictures of various lifestyle-appropriate scenes may be prepared to allow selection of a picture of a scene in response to a patient request.

For ease of explanation, the following description assumes that a left/right/both eye button 89A, an image capture/projection button 89B, and a projection start button 89C are already being displayed in the control button display region 86A4 as illustrated in FIG. 9 as an example.

The left/right/both eye button 89A is a software key pressed to select whether to project a picture into the right eye 44R only, whether to project a picture into the left eye 44L only, or whether to project a picture into both eyes. The image capture/projection button 89B is a software key pressed to select whether to execute image capture with the outward-facing camera 50 or to project a picture into the subject eye 44. The projection start button 89C is a software key pressed to instruct the start of projection of a picture into the subject eye 44.

For ease of explanation, the following description assumes that whether to project a picture into the right eye 44R only, to project a picture into the left eye 44L only, or to project a picture into both eyes has already been decided by pressing the left/right/both eye button 89A.

In the server-side processing illustrated in FIG. 6, first, at step 200, the acquisition section 100 acquires the patient information 94A from the secondary storage section 94, and then transitions to step 202. Note that the patient information 94A acquired at step 200 is, for example, displayed in the patient information display region 86A1 as illustrated in FIG. 9 under control from the CPU 90.

At step 202, the processing section 99 determines whether or not an eyewear ID has been received by the reception device 84. The eyewear ID is information enabling unique identification of the wearable terminal device 12 that is being worn by the patient.

Processing transitions to step 204 when negative determination is made at step 202, i.e. when the eyewear ID has not been received by the reception device 84. Processing transitions to step 206 when affirmative determination is made at step 202, i.e. when the eyewear ID has been received by the reception device 84.

Note that configuration may be made such that at step 202 the eyewear ID of the wearable terminal device 12 being worn by the patient is transmitted from the wearable terminal device 12 to the server device 14 through the wireless communication section 112, and the server device 14 receives the eyewear ID by causing the processing section 99 to acquire the eyewear ID through the wireless communication section 112.

At step 204, the processing section 99 determines whether or not an end condition relating to the server-side processing has been satisfied. The end condition relating to the server-side processing indicates a condition to end the server-side processing. Examples of the end condition relating to the server-side processing include a condition that a specific period of time has elapsed, a condition that the reception device 84 has received an end instruction, and/or a condition that an error requiring the server-side processing to be forcibly ended has been detected by the CPU 90.

Processing transitions to step 202 when negative determination is made at step 204, i.e. when the end condition relating to the server-side processing has not been satisfied. The server-side processing is ended when affirmative determination is made at step 204, i.e. when the end condition relating to the server-side processing has been satisfied.

At step 206, the processing section 99 determines whether or not an intraocular lens ID has been received by the reception device 84. The intraocular lens ID is information enabling unique identification of the intraocular lens due to be inserted into the subject eye 44.

Processing transitions to step 208 when negative determination is made at step 206, i.e. when the intraocular lens ID has not been received by the reception device 84. Processing transitions to step 210 when affirmative determination is made at step 206, i.e. when the intraocular lens ID has been received by the reception device 84.

At step 208, the processing section 99 determines whether or not the end condition relating to the server-side processing has been satisfied. Processing transitions to step 206 when negative determination is made at step 208, i.e. when the end condition relating to the server-side processing has not been satisfied. The server-side processing is ended when affirmative determination is made at step 208, i.e. when the end condition relating to the server-side processing has been satisfied.

At step 210, the processing section 99 acquires from the secondary storage section 94 the intraocular lens information 94B corresponding to the intraocular lens identified by the intraocular lens ID received by the reception device 84 at step 206, and then processing transitions to step 212. Note that as an example, the intraocular lens information 94B acquired at step 210 is displayed in the intraocular lens information display region 86A2 as illustrated in FIG. 9 under control from the CPU 90.

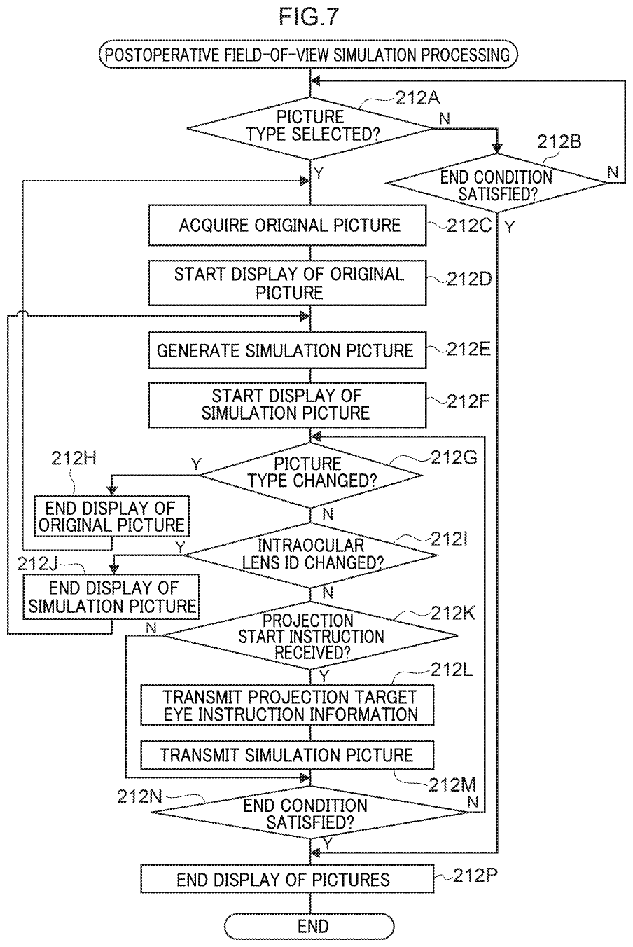

At step 212, the CPU 90 executes postoperative field-of-view simulation processing as illustrated in the example of FIG. 7, and then processing transitions to step 214.

In the postoperative field-of-view simulation processing illustrated in FIG. 7, at step 212A the processing section 99 determines whether or not the picture type has been selected. The picture type (scene) is determined to have been selected when a picture selection button 87 has been pressed on the touch panel 84A.

Processing transitions to step 212B when negative determination is made at step 212A, i.e. when the picture type has not been selected. Processing transitions to step 212C when affirmative determination is made, i.e. when the picture type has been selected.

At step 212B, the processing section 99 determines whether or not the end condition relating to the server-side processing has been satisfied. Processing transitions to step 212A when negative determination is made at step 212B, i.e. when the end condition relating to the server-side processing has not been satisfied. Processing transitions to step 212P when affirmative determination is made at step 212B, i.e. when the end condition relating to the server-side processing has been satisfied.

At step 212C, the acquisition section 100 acquires an original picture corresponding to the picture type selected at step 212A or a picture type changed at step 212G and then processing transitions to step 212D.

At step 212D, the display control section 106 controls the display 86A to start displaying the original picture acquired at step 212C on the display 86A, and then processing transitions to step 212E. The original picture acquired at step 212C is thus displayed in the original picture display region 86A5.

At step 212E, the generation section 102 generates a simulation picture by converting or performing image processing on the original picture acquired at step 212C based on the optometry information 94A2 acquired at step 200 and the intraocular lens information 94B acquired at step 210 or step 212I. The simulation picture is a simulation picture (image) reflecting how vision would appear in a case in which the intraocular lens is prescribed for the subject eye, or in other words, is a picture (image) of scenery as it would appear to the patient in a case in which the intraocular lens is prescribed and fitted to the patient.

At the next step 212F, the display control section 106 controls the display 86A such that the display 86A starts display of the simulation picture generated at step 212E. Processing then transitions to step 212G. The simulation picture generated at step 212E is thus displayed in the simulation picture display region 86A6.

At step 212G the processing section 99 determines whether or not the picture type has been changed. Changing of the picture type is achieved by pressing a picture selection button 87 corresponding to an original picture different to the currently acquired original picture.

Processing transitions to step 212H when affirmative determination is made at step 212G, i.e. when the picture type has been changed. Processing transitions to step 212I when negative determination is made at step 212G, i.e. when the picture type has not been changed.

At step 212H, the display control section 106 controls the display 86A such that display of the original picture on the display 86A is ended, and then processing transitions to step 212C. The original picture is thus cleared from the original picture display region 86A5.

At step 212I, the processing section 99 determines whether or not the intraocular lens ID has been changed. Changing of the intraocular lens ID is achieved when the reception device 84 receives an intraocular lens ID different to the intraocular lens ID corresponding to the currently acquired intraocular lens information.

Processing transitions to step 212J when affirmative determination is made at step 212I, i.e. when the intraocular lens ID has been changed. Processing transitions to step 212K when negative determination is made at step 212I, i.e. when the intraocular lens ID has not been changed. Note that when the intraocular lens ID is changed, intraocular lens information 94B corresponding to the intraocular lens identified by the changed intraocular lens ID is acquired and the intraocular lens information 94B held by the acquisition section 100 is updated.

At step 212J, the display control section 106 controls the display 86A such that the display 86A ends display of the simulation picture, and then processing transitions to step 212E. The simulation picture is thus cleared from the simulation picture display region 86A6.

At step 212K, the processing section 99 determines whether or not a projection start instruction has been received. A projection start instruction is determined to have been received when the projection start button 89C has been pressed.

Processing transitions to step 212N when negative determination is made at step 212K, i.e. a projection start instruction has not been received. Processing transitions to step 212L when affirmative determination is made at step 212K, i.e. when a projection start instruction has been received.

At step 212L, the processing section 99 transmits projection target eye instruction information to the control device 18 through the wireless communication section 82, and then processing transitions to step 212M. The projection target eye instruction information is information indicating a target eye into which to project the simulation picture. The “target eye” referred to here may be the right eye 44R, the left eye 44L, or both eyes. The right eye 44R, the left eye 44L, or both eyes are selected as the “target eye” by pressing the left/right/both eye button 89A.

At step 212M, the output section 104 transmits the simulation picture generated at step 212E to the control device 18 through the wireless communication section 82, and then processing transitions to step 212N.

At step 212N, the processing section 99 determines whether or not the end condition relating to the server-side processing has been satisfied. Processing transitions to step 212G when negative determination is made at step 212N, i.e. when the end condition relating to the server-side processing has not been satisfied. Processing transitions to step 212P when affirmative determination is made at step 212N, i.e. when the end condition relating to the server-side processing has been satisfied.

At step 212P, the display control section 106 controls the display 86A such that the display 86A ends display of the original picture and the simulation picture, and the postoperative field-of-view simulation processing is ended. Executing the processing of step 212P clears the original picture from the original picture display region 86A5, and clears the simulation picture from the simulation picture display region 86A6.

At step 214 of the server-side processing illustrated in FIG. 6, the processing section 99 performs wireless communication with the control device 18 to cause the right-eye inward-facing camera 48R and the left-eye inward-facing camera 48L to image the anterior segments of the subject eyes 44, and then processing transitions to step 216. The anterior segment of the right eye 44R is thereby imaged by the right-eye inward-facing camera 48R, and the anterior segment of the left eye 44L is thereby imaged by the left-eye inward-facing camera 48L.

In the following, for ease of explanation, an image obtained by imaging the anterior segment of the right eye 44R with the right-eye inward-facing camera 48R is referred to as a right-eye anterior segment image, and an image obtained by imaging the anterior segment of the left eye 44L with the left-eye inward-facing camera 48L is referred to as a left-eye anterior segment image.

At step 216, the processing section 99 performs wireless communication with the control device 18 to cause the control section 144 to detect the inter-pupil distance based on the right-eye anterior segment image and the left-eye anterior segment image, and then processing transitions to step 218. The inter-pupil distance referred to here indicates the distance between the pupil in the anterior segment of the right eye 44R represented by the right-eye anterior segment image and the pupil in the anterior segment of the left eye 44L represented by the left-eye anterior segment image.

At step 218, the processing section 99 performs wireless communication with the control device 18 to cause the control section 144 to adjust the positions of the reflection mirrors 42 based on the eyewear ID received at step 202, the inter-pupil distance detected at step 216, and the like. The processing section 99 also performs wireless communication with the control device 18 to cause the control section 144 to control the scanner 28 so as to correct the optical axes of the laser beams based on the eyewear ID received at step 202, the inter-pupil distance detected at step 216, and the like, and to perform home positioning.

Note that the positions of the reflection mirrors 42 are adjusted by controlling the mirror drive sources 72 with the control section 144. The correction of the optical axes of the laser beams and the home positioning is achieved by controlling the scanner 28 with the control section 144.

Whether or not the evaluation of the patient is determined to be positive is based on whether or not the patient presses the response button 19 in response to questioning by the medical service professional. When the patient presses the response button 19, response information indicating that the response button 19 has been pressed is transmitted from the control device 18 to the server device 14 through the wireless communication section 112, and the response information is received by the wireless communication section 82 of the server device 14. Note that when the response information is received by the wireless communication section 82 of the server device 14, the CPU 90 may display a message, image, or the like on the display 86A to indicate that the response information has been received by the wireless communication section 82.

Processing transitions to step 206 when the verdict of the patent is not positive at step 220. The server-side processing is ended when affirmative determination is made at step 220, i.e. when the verdict of the patient is positive.

Note that the verdict of the patient regarding the simulation picture projected onto the retina is made through communication between the medical service professional and the patient to confirm how vision appears. The medical service professional judges whether or not a specified intraocular lens is appropriate for the patient. If the verdict of the patient is not positive, a new intraocular lens ID is specified by the medical service professional, and the simulation picture is displayed again.

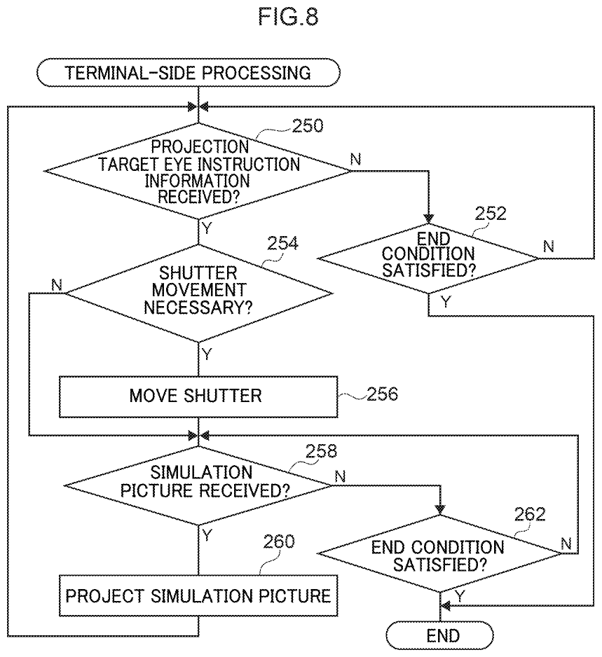

Next, explanation follows regarding terminal-side processing implemented by the CPU 120 executing the terminal-side program 124A when a main power source (not illustrated in the drawings) for the wearable terminal device 12 is turned on, with reference to FIG. 8. Note that the terminal-side processing illustrated in FIG. 8 is processing performed for a patient prior to surgery in order for the patient to confirm how their vision will appear following surgery.

In the terminal-side processing illustrated in FIG. 8, first at step 250, the processing section 142 determines whether or not the projection target eye instruction information transmitted as a result of executing the processing of step 212L in the postoperative field-of-view simulation processing has been received by the wireless communication section 112. Processing transitions to step 252 when negative determination is made at step 250, i.e. when the projection target eye instruction information has not been received by the wireless communication section 112. Processing transitions to step 254 when affirmative determination is made at step 250, i.e. when the projection target eye instruction information has been received by the wireless communication section 112.

At step 252, the processing section 142 determines whether or not an end condition relating to the terminal-side processing has been satisfied. The end condition relating to the terminal-side processing indicates a condition to end the terminal-side processing. Examples of the end condition relating to the terminal-side processing include a condition that a specific period of time has elapsed, a condition that an end instruction has been received by the reception device 84, and/or a condition that an error requiring the terminal-side processing to be forcibly ended has been detected by the CPU 120.

Processing transitions to step 250 when negative determination is made at step 252, i.e. when the end condition relating to the terminal-side processing has not been satisfied. The terminal-side processing is ended when affirmative determination is made at step 252, i.e. when the end condition relating to the terminal-side processing has been satisfied.

At step 254, the processing section 142 determines whether or not there is a need to move at least one of the right-eye shutter 121R or the left-eye shutter 121L, based on the projection target eye instruction information received by the wireless communication section 112.

Processing transitions to step 258 when negative determination is made at step 254, i.e. when there is no need to move either the right-eye shutter 121R or the left-eye shutter 121L. Processing transitions to step 256 when affirmative determination is made at step 254, i.e. when there is a need to move at least one of the right-eye shutter 121R or the left-eye shutter 121L.

At step 256, the control section 144 moves the shutter 121 based on the projection target eye instruction information received by the wireless communication section 112.

For example, when a simulation picture is to be projected onto the retinas 46 of both eyes, the first shutter drive source 134R and the second shutter drive source 134L are controlled such that the right-eye shutter 121R is disposed at the first position P1, and the left-eye shutter 121L is disposed at the third position P3. When a simulation picture is to be projected onto the retina 46R, the first shutter drive source 134R and the second shutter drive source 134L are controlled such that the right-eye shutter 121R is disposed at the first position P1 and the left-eye shutter 121L is disposed at the fourth position P4. Furthermore, when a simulation picture is to be projected onto the retina 46L, the first shutter drive source 134R and the second shutter drive source 134L are controlled such that the right-eye shutter 121R is disposed at the second position P2 and the left-eye shutter 121L is disposed at the third position P3.

In cases in which the shutters are not mechanically but electrically driven, the transmissivity/non-transmissivity of laser beams by the left and right shutters may be controlled based on the projection target eye instruction information.

At the next step 258, the processing section 142 determines whether or not a simulation picture transmitted as a result of executing the processing of step 212M included in the postoperative field-of-view simulation processing has been received.

Processing transitions to step 260 when affirmative determination is made at step 258, i.e. when the simulation picture has been received. Processing transitions to step 262 when negative determination is made at step 258, i.e. when the simulation picture has not been received.

At step 260, the control section 144 causes the laser light source 114 to emit a laser beam according to the simulation picture received by the wireless communication section 112, and controls the scanner 28 according to the projection target eye instruction information so as to project the simulation picture onto the retina 46.

At the next step 262, the processing section 142 determines whether or not the end condition relating to the terminal-side processing has been satisfied. Processing transitions to step 258 when negative determination is made at step 262, i.e. when the end condition relating to the terminal-side processing has not been satisfied. The terminal-side processing is ended when affirmative determination is made at step 262, i.e. when the end condition relating to the terminal-side processing has been satisfied.

As described above, the ophthalmic system 10 is equipped with the wearable terminal device 12 and the server device 14 that generates the simulation picture by converting the original picture into the simulation picture based on the optometry information 94A2 and the intraocular lens information 94B. The wearable terminal device 12 projects the simulation picture generated by the server device 14 onto the retina 46. The ophthalmic system 10 is thus capable of allowing the patient to directly experience and confirm how vision will actually appear following surgery.

The wearable terminal device 12 is equipped with the optical system 27 that guides laser beams onto the retina 46, and the wireless communication section 112 that receives the simulation picture generated by the server device 14 based on the optometry information 94A2 and the intraocular lens information 94B. The wearable terminal device 12 also includes the control section 144 that controls the laser light source 114 and the optical system 27 so as to project the simulation picture received by the wireless communication section 112 onto the retina 46. The wearable terminal device 12 is thus capable of allowing the patient to directly experience and confirm how vision will actually appear following surgery.

The wearable terminal device 12 is also equipped with the scanner 28 to scan the laser beams, and the reflection mirrors 42 to reflect the laser beams scanned by the scanner 28 onto the retinas 46. Thus even for patients with cataracts, namely, patients whose crystalline lenses are cloudy, the wearable terminal device 12 is capable of presenting the patient with how their vision will appear following surgery.

Moreover, the wearable terminal device 12 is also equipped with the right-eye inward-facing camera 48R and the left-eye inward-facing camera 48L to image the anterior segments of the subject eyes 44. The control section 144 then detects the inter-pupil distance based on the right-eye anterior segment image and the left-eye anterior segment image obtained by imaging with the right-eye inward-facing camera 48R and the left-eye inward-facing camera 48L, and controls the positions of the reflection mirrors 42 based on the detected inter-pupil distance. The wearable terminal device 12 thereby enables patients with different inter-pupil distances to directly experience and confirm how vision will actually appear following surgery.

The wearable terminal device 12 is also equipped with the right-eye optical system 27R and the left-eye optical system 27L. The wearable terminal device 12 is further equipped with the laser beam splitter 20 that splits a laser beam into a laser beam for the right-eye optical system 27R and a laser beam for the left-eye optical system 27L. Thus the wearable terminal device 12 enables simulation pictures to be projected into both eyes at the same time using the single laser light source 114.

The wearable terminal device 12 is also equipped with the outward-facing camera 50. The control section 144 controls the wireless communication section 112 to transmit an image obtained by imaging with the outward-facing camera 50 to the server device 14. The wearable terminal device 12 is thus capable of allowing the patient to directly experience and confirm how the actual external field of vision would appear following surgery.

The wearable terminal device 12 is equipped with the eyewear terminal device 16 including the optical system 27. The wearable terminal device 12 is thus capable of projecting the simulation picture while the patient is wearing the eyewear terminal device 16.

The server device 14 is equipped with the generation section 102 that generates the simulation picture by converting the original picture based on the optometry information 94A2 and the intraocular lens information 94B. The server device 14 is also equipped with the output section 104 that outputs the simulation picture generated by the generation section 102 to the wearable terminal device 12 by performing wireless communication with the wearable terminal device 12. The server device 14 is thus capable of allowing the patient to directly experience and confirm how vision will actually appear following surgery.

The server device 14 is further equipped with the acquisition section 100 that acquires the original picture from the plural original pictures of different scenes in response to an instruction received by the reception device 84. The generation section 102 generates the simulation picture by converting the original picture acquired by the acquisition section 100 based on the optometry information 94A2 and the intraocular lens information 94B. The server device 14 is thus capable of projecting a simulation picture relevant to the preferences or lifestyle of the patient onto the retina 46 of the patient using the wearable terminal device 12.

The server device 14 is further equipped with the display control section 106 that controls the display 86A such that the original picture is displayed in the original picture display region 86A5, and the simulation picture is displayed in the simulation picture display region 86A6. The server device 14 is thus capable of allowing the medical service professional to visually acknowledge both the original picture and the simulation picture.

Note that although pictures have been given as an example in the exemplary embodiment described above, the technology disclosed herein is not limited thereto, and instead of pictures, still images may be employed, or slide images made up of plural still images may be employed.

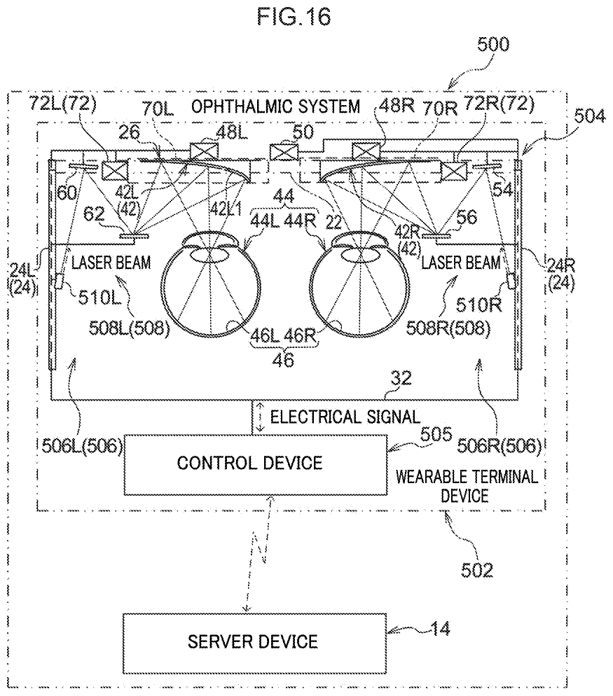

Moreover, although the laser beam splitter 20 and the laser light source 114 have been given as examples in the exemplary embodiment described above, the technology disclosed herein is not limited thereto. For example, an ophthalmic system 500 as illustrated in FIG. 16 may be employed instead of the ophthalmic system 10.

The ophthalmic system 500 differs from the ophthalmic system 10 in that a wearable terminal device 502 is provided instead of the wearable terminal device 12. The wearable terminal device 502 differs from the wearable terminal device 12 in that the optical fibers 30, 38, 40 and the laser beam splitter 20 are not provided. The wearable terminal device 502 further differs from the wearable terminal device 12 in that a control device 505 is provided instead of the control device 18. The wearable terminal device 502 also differs from the wearable terminal device 12 in that an eyewear terminal device 504 is provided instead of the eyewear terminal device 16. The control device 505 differs from the control device 18 in that the laser light source 114 is not provided.

The eyewear terminal device 504 further differs from the eyewear terminal device 16 in that an optical system 506 is provided instead of the optical system 27. The optical system 506 differs from the optical system 27 in that in that it includes a right-eye optical system 508R instead of the right-eye optical system 27R, and in that it includes a left-eye optical system 508L instead of the left-eye optical system 27L. Moreover, the optical system 506 differs from the optical system 27 in that a scanner 508 is provided instead of the scanner 28.

The scanner 508 differs from the scanner 28 in that it includes a right-eye scanner 508R instead of the right-eye scanner 28R, and it includes a left-eye scanner 508L instead of the left-eye scanner 28L.

The right-eye scanner 508R differs from the right-eye scanner 28R in that light is scanned from a right-eye laser light source 510R instead of scanning laser beams from the right-eye illumination section 52. The right-eye laser light source 510R is an example of a right-eye light source according to the technology disclosed herein, and is employed in the right-eye optical system 508R. The right-eye laser light source 510R emits a laser beam towards the MEMS mirror 54 similarly to the right-eye illumination section 52. The right-eye laser light source 510R is connected to the bus line 32 and operates under control from the CPU 120.

The left-eye scanner 510L differs from the left-eye scanner 28L in that light is scanned from a right-eye laser light source 510L instead of scanning laser beams from the left-eye illumination section 58. The left-eye laser light source 510L is an example of a left-eye light source according to the technology disclosed herein, and is employed in the left-eye optical system 508L. The left-eye laser light source 510L emits a laser beam towards the MEMS mirror 60 similarly to the left-eye illumination section 58. The left-eye laser light source 510L is connected to the bus line 32 and operates under control from the CPU 120.

The wearable terminal device 502 thereby renders the optical fibers 30, 38, 40 and the laser beam splitter 20 redundant, enabling a contribution to be made to greater compactness of the wearable terminal device 502.

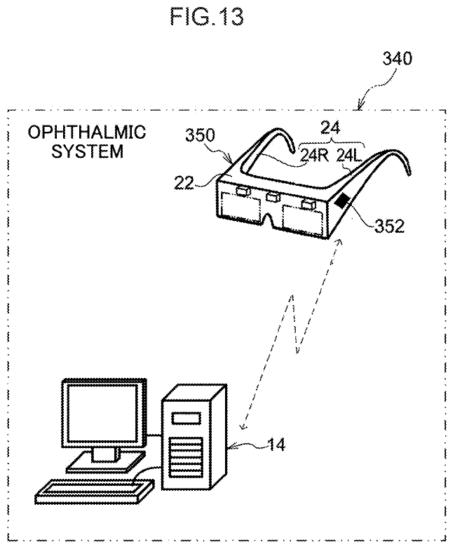

Although the right-eye laser light source 510R, the left-eye laser light source 510L, the right-eye optical system 508R, and the left-eye optical system 508L are attached to the eyewear terminal device 504 in the example illustrated in FIG. 16, the technology disclosed herein is not limited thereto. For example, similarly to in the example illustrated in FIG. 13, a device with functionality corresponding to that of the control device 505 may be attached to a frame of the eyewear terminal device 504.

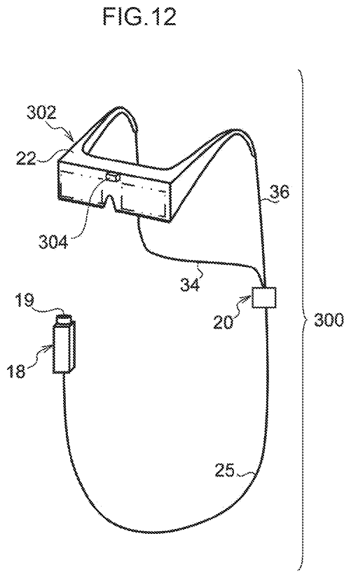

Although the right-eye inward-facing camera 48R, the left-eye inward-facing camera 48L, and the outward-facing camera 50 are given as examples in the exemplary embodiment described above, the technology disclosed herein is not limited thereto. For example, as illustrated in FIG. 12, a wearable terminal device 300 may be employed instead of the wearable terminal device 12. The wearable terminal device 300 differs from the wearable terminal device 12 in that an eyewear terminal device 302 is provided instead of the eyewear terminal device 16. The eyewear terminal device 302 differs from the eyewear terminal device 16 in that an inward/outward-facing camera 304 is employed instead of the right-eye inward-facing camera 48R, the left-eye inward-facing camera 48L, and the outward-facing camera 50. The inward/outward-facing camera 304 is a camera configured by integrating together an inward-facing camera capable of imaging the anterior segment of the right eye 44R and the anterior segment of the left eye 44L at the same time and an outward-facing camera having similar functionality to that of the outward-facing camera 50.

Although the right-eye inward-facing camera 48R, the left-eye inward-facing camera 48L, and the outward-facing camera 50 are disposed on the outside of the rim piece 22 in the exemplary embodiment described above, the technology disclosed herein is not limited thereto. For example, the right-eye inward-facing camera 48R, the left-eye inward-facing camera 48L, and the outward-facing camera 50 may be embedded in the rim piece 22 such that respective imaging lenses thereof (not illustrated in the drawings) are exposed toward the imaging subjects.

Although an example is given in the exemplary embodiment described above of the wearable terminal device 12 in which the control device 18 and the laser beam splitter 20 are external to the eyewear terminal device 16, the technology disclosed herein is not limited thereto. For example, an ophthalmic system 340 as illustrated in FIG. 13 may be employed instead of the ophthalmic system 10.

The ophthalmic system 340 differs from the ophthalmic system 10 in that it does not include the control device 18, the laser beam splitter 20, nor the cables 25, 34, 36. The ophthalmic system 340 also differs from the ophthalmic system 10 in that it includes an eyewear terminal device 350 instead of the eyewear terminal device 16.

The eyewear terminal device 350 includes a controller 352 configured by integrating together a device with functionality equivalent to that of the control device 18 and a device with functionality equivalent to that of the laser beam splitter 20. The controller 352 is housed in the left temple piece 24L. In such a configuration cables equivalent to the cables 34, 36 are also housed in the frame of the eyewear terminal device 350. The frame of the eyewear terminal device 350 referred to here indicates, for example, the rim piece 22 and the temple piece 24.

The controller 352 may be provided in the right temple piece 24R. Moreover, a configuration may be adopted in which a device with functionality equivalent to that of the control device 18 and a device with functionality equivalent to that of the laser beam splitter 20 are separately housed in the frame of the eyewear terminal device 350. In such cases, a cable equivalent to the cable 25, namely, a cable connecting together the device with functionality equivalent to that of the control device 18 and the device with functionality equivalent to that of the laser beam splitter 20, is also housed in the frame of the eyewear terminal device 350.

Moreover, although the shutters 121 have been given as an example in the exemplary embodiment described above, the technology disclosed herein is not limited thereto, and, instead of the shutters 121, a device may be employed that is capable of being controlled so as to let light pass through, such as a liquid crystal shutter.

Moreover, although laser beams have been given as examples in the exemplary embodiment described above, the technology disclosed herein is not limited thereto, and, for example, light from super luminescent diodes may be employed instead of laser beams.

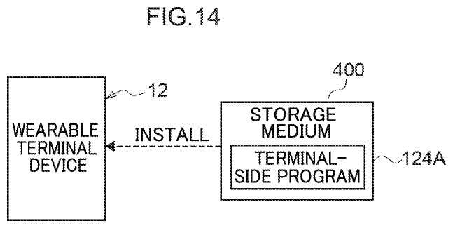

Moreover, although examples have been given in the exemplary embodiments described above in which the terminal-side program 124A is read from the secondary storage section 124, the terminal-side program 124A does not necessarily have to be initially stored on the secondary storage section 124. For example, as illustrated in FIG. 14, a configuration may be adopted in which the terminal-side program 124A is first stored on a freely selected portable storage medium 400 such as an SSD, USB memory, or DVD-ROM or the like. In such a configuration the terminal-side program 124A on the storage medium 400 is then installed on the wearable terminal device 12, and the installed terminal-side program 124A then executed by the CPU 120.

Moreover, a configuration may be adopted in which the terminal-side program 124A is stored on a storage section of another computer or server device or the like connected to the wearable terminal device 12 over a communication network (not illustrated in the drawings), such that the terminal-side program 124A is downloaded and then installed in response to a request from the wearable terminal device 12. In such a configuration, the installed terminal-side program 124A is then executed by the CPU 120.

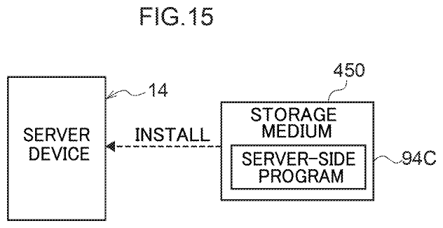

Moreover, although explanation has been given in the exemplary embodiment described above in which the server-side program 94C is read from the secondary storage section 94, the server-side program 94C does not necessarily have to be initially stored on the secondary storage section 94. For example, a configuration may be adopted in which, as illustrated in FIG. 15, the server-side program 94C is first stored on a freely selected portable storage medium 450 such as an SSD, USB memory, or DVD-ROM or the like. In such a configuration the server-side program 94C on the storage medium 450 is then installed on the server device 14, and the installed server-side program 94C is then executed by the CPU 90.

Moreover, a configuration may be adopted in which the server-side program 94C is stored on a storage section of another computer or server device or the like connected to the server device 14 over a communication network (not illustrated in the drawings), such that the server-side program 94C is downloaded and then installed in response to a request from the server device 14. In such a configuration, the installed server-side program 94C is then executed by the CPU 90.

Moreover, the server-side processing and the terminal-side processing in the exemplary embodiment described above are merely given as examples thereof. Thus obviously steps that are not required may be removed, new steps may be added, and the sequence of processing may be reordered within a range not departing from the spirit thereof.

Moreover, although examples are given in the exemplary embodiment described above of cases in which the server-side processing and the terminal-side processing are implemented by a software configuration utilizing a computer, the technology disclosed herein is not limited thereto. For example, instead of a software configuration utilizing a computer, at least one type of processing of the server-side processing or the terminal-side processing may be executed by a purely hardware configuration, i.e. a FPGA, ASIC, configuration or the like. At least one type of processing of the server-side processing or the terminal-side processing may also be executed by a configuration combining a software configuration and a hardware configuration.

All cited documents, patent applications, and technical standards mentioned in the present specification are incorporated by reference in the present specification to the same extent as if each individual cited document, patent application, or technical standard was specifically and individually indicated to be incorporated by reference.

The following supplements are also disclosed in relation to the above exemplary embodiment.

Supplement 1

An ophthalmic instrument (12, 300, 502) including:

a light source (114, 510R, 510L);

an optical system (27, 506) that guides light emitted from the light source (114, 510R, 510L) onto a retina (46) of a subject eye (44);

a communication section (112) that receives a simulation image generated by an image generation device (14) based on optometry information (94A2) for the subject eye (44) and intraocular lens information (94B) relating to an intraocular lens prescribable for the subject eye (44), the simulation image corresponding to how vision would appear in a case in which the intraocular lens is prescribed for the subject eye (44); and

a control section (144) that controls the light source (114, 510R, 510L) and the optical system (27, 506) such that the simulation image received by the communication section (112) is projected onto the retina (46).

Supplement 2

The ophthalmic instrument (12, 300, 502) of supplement 1, wherein the simulation image is an image obtained by performing image processing on an original image based on the optometry information (94A2) and the intraocular lens information (94B).

Supplement 3

The ophthalmic instrument (12, 300, 502) of supplement 2, wherein the original image and the simulation image are moving images.

Supplement 4

The ophthalmic instrument (12, 300, 502) of either supplement 2 or supplement 3, wherein the original image is an image selected from plural images of different scenes.

Supplement 5

The ophthalmic instrument (12, 300, 502) of any one of supplement 1 to supplement 4, wherein the optical system (27, 506) includes a scanner (28, 508) that scans the light and a reflection member (42) that reflects light scanned by the scanner (28, 508) toward the retina (46).

Supplement 6

The ophthalmic instrument (12, 300, 502) of supplement 5, further including an anterior segment camera (48R, 48L, 304) that images an anterior segment of the subject eye (44), wherein:

the control section (144) detects an inter-pupil distance based on an anterior segment image obtained by imaging with the anterior segment camera (48R, 48L, 304), and controls a position of the reflection member (42) based on the detected inter-pupil distance.

Supplement 7

The ophthalmic instrument (12) of any one of supplement 1 to supplement 6, wherein:

the optical system (27) includes a right-eye optical system (27R) to guide the light onto the retina (46R) of a right eye (44R), and a left-eye optical system (27L) to guide the light onto the retina (46L) of a left eye (44L); and

the ophthalmic instrument (12) further includes an optical splitter (20) that splits the light into light for the right-eye optical system (27R) and light for the left-eye optical system (27L).

Supplement 8

The ophthalmic instrument of any one of supplement 1 to supplement 7, wherein: the optical system (506) includes a right-eye optical system (27R) to guide the light onto the retina (46R) of a right eye (44R), and a left-eye optical system (27L) to guide the light onto the retina (46L) of a left eye (44L); and

the light source (510R, 510L) includes a right-eye light source (510R) employed with the right-eye optical system (27R), and a left-eye light source (510L) employed with the left-eye optical system (27L).

Supplement 9

The ophthalmic instrument (12, 300, 502) of any one of supplement 1 to supplement 8, further including a field of vision camera (50, 304) that images an external field of vision, wherein:

the control section (144) controls the communication section (112) such that a field of vision image obtained by imaging with the field of vision camera (50, 304) is transmitted to an image generation device (14).

Supplement 10

The ophthalmic instrument (12, 300, 502) of supplement 9, wherein the simulation image is an image obtained by performing image processing on the field of vision image based on the optometry information (94A2) and the intraocular lens information (94B).

Supplement 11

The ophthalmic instrument (12, 300, 502) of any one of supplement 1 to supplement 10, further including an eyewear terminal device (16, 302, 350, 504) provided with at least the optical system (27, 506) from the light source (114, 510R, 510L), the optical system (27, 506), the communication section (112), and the control section (144).

Supplement 12

An image generation device (14) including:

a generation section (102) that generates a simulation image based on optometry information (94A2) for a subject eye (44) and intraocular lens information (94B) relating to an intraocular lens prescribable for the subject eye (44), the simulation image corresponding to how vision would appear in a case in which the intraocular lens is prescribed for the subject eye (44); and

an output section (104) that outputs the simulation image generated by the generation section to a projection device.

Supplement 13

The ophthalmic instrument (12, 300, 502) of supplement 12, wherein the simulation image is an image obtained by performing image processing on an original image based on the optometry information (94A2) and the intraocular lens information (94B).

Supplement 14

The ophthalmic instrument (12, 300, 502) of supplement 13, wherein the original image and the simulation image are moving images.

Supplement 15

The image generation device (14) of either supplement 13 or supplement 14, further including an acquisition section (100) that acquires an image from plural images of different scenes as the original image in response to an instruction received by a reception section (84).

Supplement 16

The image generation device (14) of any one of supplement 12 to supplement 15, further including:

a display section (86A) that displays an image; and

a display control section (106) that controls the display section (86A) so as to display the original image and the simulation image on the display section (86A).

Supplement 17

A program (124A) that causes a computer (110) to function as the control section (144) included in the ophthalmic instrument (12, 300, 502) of any one of supplement 1 to supplement 11.

Supplement 18

A program (94C) that causes a computer (80) to function as the generation section (102) and the output section (104) included in the image generation device (14) of any one of supplement 12 to supplement 16.

Supplement 19

An ophthalmic system (10, 300, 340, 500) including:

a projection device (12, 300, 502) that projects an image onto a retina (46) of a subject eye (44); and

an image generation device (14) that generates a simulation image based on optometry information (94A2) for the subject eye (44) and intraocular lens information (94B) relating to an intraocular lens prescribable for the subject eye (44), the simulation image corresponding to how vision would appear in a case in which the intraocular lens is prescribed for the subject eye (44), wherein

the projection device (12, 300, 502) projects the simulation image generated by the image generation device (12, 300, 502) onto the retina (46).

### EXPLANATION OF THE REFERENCE NUMERALS

10, 300, 340, 500 ophthalmic system
12 wearable terminal device
14 server device
16, 302, 350, 504 eyewear terminal device
20 laser beam splitter
27, 506 optical system
27R, 506R right-eye optical system
27L, 506L left-eye optical system
28, 508 scanner
42 reflection mirror
44 subject eye
46 retina
48R right-eye inward-facing camera
48L left-eye inward-facing camera
50 outward-facing camera
82, 112 wireless communication section
84 reception device
86A display
90, 120 CPU
94A2 optometry information
94B intraocular lens information
94C server-side program
100 acquisition section
102 generation section
104 output section
106 display control section 114
124A terminal-side program
144 control section
304 inward/outward-facing camera
510R right-eye laser light source
510L left-eye laser light source

1. An ophthalmic instrument comprising:
a light source;
an optical system that guides light emitted from the light source onto a retina of a subject eye;
a communication section that receives a simulation image generated based on optometry information for the subject eye and intraocular lens information relating to an intraocular lens prescribable for the subject eye, the simulation image corresponding to how vision would appear in a case in which the intraocular lens is prescribed for the subject eye; and
a control section that controls the light source and the optical system such that the simulation image received by the communication section is projected onto the retina.

2. The ophthalmic instrument of claim 1, wherein the simulation image is an image obtained by performing image processing on an original image based on the optometry information and the intraocular lens information.

3. The ophthalmic instrument of claim 2, wherein the original image and the simulation image are moving images.

4. The ophthalmic instrument of claim 2, wherein the original image is an image selected from a plurality of images of different scenes.

5. The ophthalmic instrument of claim 1, wherein the optical system includes a scanner that scans the light and a reflection member that reflects light scanned by the scanner toward the retina.

6. The ophthalmic instrument of claim 5, further comprising an anterior segment camera that images an anterior segment of the subject eye, wherein:
the control section detects an inter-pupil distance based on an anterior segment image obtained by imaging with the anterior segment camera, and controls a position of the reflection member based on the detected inter-pupil distance.

7. The ophthalmic instrument of claim 1, wherein:
the optical system includes a right-eye optical system to guide the light onto the retina of a right eye, and a left-eye optical system to guide the light onto the retina of a left eye; and
the ophthalmic instrument further comprises an optical splitter that splits the light into light for the right-eye optical system and light for the left-eye optical system.

8. The ophthalmic instrument of claim 1, wherein:
the optical system includes a right-eye optical system to guide the light onto the retina of a right eye, and a left-eye optical system to guide the light onto the retina of a left eye; and
the light source includes a right-eye light source employed with the right-eye optical system, and a left-eye light source employed with the left-eye optical system.

9. The ophthalmic instrument of claim 1, further comprising a field of vision camera that images an external field of vision, wherein:
the control section controls the communication section such that a field of vision image obtained by imaging with the field of vision camera is transmitted to an image generation device.

10. The ophthalmic instrument of claim 9, wherein the simulation image is an image obtained by performing image processing on the field of vision image based on the optometry information and the intraocular lens information.

11. The ophthalmic instrument of claim 1, further comprising an eyewear terminal device provided with at least the optical system from the light source, the optical system, the communication section, and the control section.

12. An image generation device comprising:
a generation section that generates a simulation image based on optometry information for a subject eye and intraocular lens information relating to an intraocular lens prescribable for the subject eye, the simulation image corresponding to how vision would appear in a case in which the intraocular lens is prescribed for the subject eye; and
an output section that outputs the simulation image generated by the generation section to a projection device.

13. The image generation device of claim 12, wherein the simulation image is an image obtained by performing image processing on an original image based on the optometry information and the intraocular lens information.

14. The image generation device of claim 13, wherein the original image and the simulation image are moving images.

15. The image generation device of claim 13, further comprising an acquisition section that acquires an image from a plurality of images of different scenes as the original image in response to an instruction received by a reception section.

16. The image generation device of claim 14, further comprising:
a display section that displays an image; and
a display control section that controls the display section so as to display the original image and the simulation image on the display section.

17. A program that causes a computer to function as the control section included in the ophthalmic instrument of claim 1.

18. A program that causes a computer to function as the generation section and the output section included in the image generation device of claim 12.

19. An ophthalmic system comprising:
a projection device that projects an image onto a retina of a subject eye; and
an image generation device that generates a simulation image based on optometry information for the subject eye and intraocular lens information relating to an intraocular lens prescribable for the subject eye, the simulation image corresponding to how vision would appear in a case in which the intraocular lens is prescribed for the subject eye, wherein
the projection device projects the simulation image generated by the image generation device onto the retina.
